(19) United States
(12) Patent Application Publication
(10) Pub. No.: US 2022/0189617 A1
(43) Pub. Date: Jun. 16, 2022

(54) METHOD AND SYSTEM FOR PROVIDER NETWORK OPTIMIZATION BASED ON IDENTIFICATION OF HIGH-PRIORITY AREAS WITH TARGETED POPULATIONS BY A HEALTH ECONOMICS APPROACH

(71) Applicant: KONINKLIJKE PHILIPS N.V., EINDHOVEN (NL)

(72) Inventors: Christoph Tobias WIRTH, Vellmar (DE); Dieter Maria Alfons VAN DE CRAEN, Balen (BE); Daniele DE MASSARI, Eindhoven (NL); Steffen Clarence PAUWS, Eindhoven (NL)

(21) Appl. No.: 17/437,964

(22) PCT Filed: Mar. 11, 2020
(86) PCT No.: PCT/EP2020/056405

§ 371 Date: Sep. 10, 2021

Related U.S. Application Data
(60) Provisional application No. 62/816,946, filed on Mar. 12, 2019.

(51) Int. Cl. G16H 40/20

(52) U.S. Cl. CPC G16H 40/20

(57) ABSTRACT

Various embodiments relate to a method for managing healthcare resources including receiving information selecting a first outcome perspective, calculating first impactibility scores for the first outcome perspective, determining a first subarea based on the first impactibility scores, and designating an allocation of healthcare resources and cost for the first subarea based on the first outcome perspective. The first impactibility scores are calculated for respective subareas including the first subarea, and the first outcome perspective corresponds to a first ratio of healthcare resources and cost.

### TECHNICAL FIELD

This disclosure relates generally to processing information, and more specifically, but not exclusively, to managing the allocation and cost of providing healthcare resources.

### BACKGROUND

As healthcare organizations shift towards value-based care, there are stronger incentives to reduce unnecessary costs. Many costs may be avoided by improving access to ambulatory services and other forms of care. However, there is a trade-off between what measures can be taken to improve access to care and the costs associated with taking those measures. Healthcare organizations do not have unlimited resources and therefore must make decisions on where to focus their efforts in order to reduce avoidable costs.

Presently, no good way exists for making informed decisions on the allocation and cost of providing healthcare resources. As a result, costs remain high and, in some cases, a substantial portion of the costs is borne by the patients receiving the care.

### SUMMARY

A brief summary of various example embodiments is presented. Some simplifications and omissions may be made in the following summary, which is intended to highlight and introduce some aspects of the various example embodiments, but not to limit the scope of the invention.

Detailed descriptions of example embodiments adequate to allow those of ordinary skill in the art to make and use the inventive concepts will follow in later sections.

Various embodiments relate to a method for managing healthcare resources, including-receiving information selecting a first outcome perspective; calculating first impactibility scores for the first outcome perspective; determining a first subarea based on the first impactibility scores; and designating an allocation of healthcare resources and cost for the first subarea based on the first outcome perspective, wherein the first impactibility scores are calculated for respective subareas including the first subarea and wherein the first outcome perspective corresponds to a first ratio of healthcare resources and cost.

Various embodiments are described, wherein generating the first impactibility scores includes: calculating second impactibility scores for respective ones of the subareas; calculating third impactibility scores for respective ones of the subareas; and calculating the first impactibility scores based on the second impactibility scores and the third impactibility scores.

Various embodiments are described, further including: applying a first weight to the second impactibility scores; applying a second weight to the third impactibility scores; and calculating the first impactibility scores based on the first weight applied to second impactibility scores and the third weight applied to the third impactibility scores, the first weight related to the second weight based on the first ratio.

Various embodiments are described, further including: calculating each of the second impactibility scores based on a gain in saved health units for a respective one of the subareas and for a number of episodes; and calculating each of the third impactibility scores based on condition-specific actually observed or estimated clinically preventable cost for a respective one of the subareas and for the number of episodes.

Various embodiments are described, wherein at least one of the second impactibility scores and the second impactibility scores is calculated based on at least one condition, the at least one condition determined to cause avoidable costs in a provider network.

Various embodiments are described, further including: determining the condition-specific actually observed or estimated clinically preventable cost for a respective one of the subareas and for the number of episodes based on cost and utilization data.

Various embodiments are described, wherein the first subarea is determined based on a greatest one of the first impactibility scores.

Various embodiments are described, further including: determining one or more potentially avoidable costs; and designating the allocation of healthcare resources and costs based on a reduction of the one or more potentially avoidable costs.

Various embodiments are described, further including: receiving information selecting a second outcome perspective; calculating the first impactibility scores for the second outcome perspective; designating an allocation of healthcare resources and cost for the first subarea based on the second outcome perspective, wherein the first impactibility scores are calculated for the respective subareas including the first subarea and wherein the second outcome perspective corresponds to a second ratio of healthcare resources and cost different from the first ratio.

Various embodiments are described, further including: comparing the first impactibility scores generated for the first outcome perspective and the first impactibility scores generated for the second outcome perspective; and selecting the first impactibility scores generated for the first outcome perspective.

Further various embodiments relate to a system for managing healthcare resources, including: an interface; and a processor configured to receive information selecting a first outcome perspective, calculate first impactibility scores for the first outcome perspective, determine a first subarea based on the first impactibility scores, and output information through the interface indicative of a designation of an allocation of healthcare resources and cost for the first subarea based on the first outcome perspective, wherein the processor is configured to calculate the first impactibility scores for respective subareas including the first subarea and wherein the first outcome perspective corresponds to a first ratio of healthcare resources and cost.

Various embodiments are described, wherein processor is configured to: calculate second impactibility scores for respective ones of the subareas; calculate third impactibility scores for respective ones of the subareas; and calculate the first impactibility scores based on the second impactibility scores and the third impactibility scores.

Various embodiments are described, wherein the processor is configured to: apply a first weight to the second impactibility scores; apply a second weight to the third impactibility scores; and calculate the first impactibility scores based on the first weight applied to second impactibility scores and the third weight applied to the third impactibility scores, the first weight related to the second weight based on the first ratio.

Various embodiments are described, wherein the processor is configured to: calculate each of the second impactibility scores based on a gain in saved health units for a respective one of the subareas and for a number of episodes; and calculate each of the third impactibility scores based on condition-specific actually observed or estimated clinically preventable cost for a respective one of the subareas and for the number of episodes.

Various embodiments are described, wherein at least one of the second impactibility scores and the second impactibility scores is calculated based on at least one condition, the at least one condition determined to cause avoidable costs in a provider network.

Various embodiments are described, wherein the processor is configured to: receive information selecting a second outcome perspective; calculate the first impactibility scores for the second outcome perspective; designate an allocation of healthcare resources and cost for the first subarea based on the second outcome perspective, wherein the first impactibility scores are calculated for the respective subareas including the first subarea and wherein the second outcome perspective corresponds to a second ratio of healthcare resources and cost different from the first ratio.

Various embodiments are described, wherein the processor is configured to: compare the first impactibility scores generated for the first outcome perspective and the first impactibility scores generated for the second outcome perspective; and select the first impactibility scores generated for the first outcome perspective.

Further various embodiments relate to a non-transitory machine-readable storage medium encoded with instructions for causing a processor to: receive information selecting a first outcome perspective; calculate first impactibility scores for the first outcome perspective, determine a first subarea based on the first impactibility scores, and output information through the interface indicative of a designation of an allocation of healthcare resources and cost for the first subarea based on the first outcome perspective, wherein the instructions are to cause the processor to calculate the first impactibility scores for respective subareas including the first subarea and wherein the first outcome perspective corresponds to a first ratio of healthcare resources and cost.

Various embodiments are described, wherein the instructions are to cause the processor to: calculate second impactibility scores for respective ones of the subareas; calculate third impactibility scores for respective ones of the subareas; and calculate the first impactibility scores based on the second impactibility scores and the third impactibility scores.

Various embodiments are described, wherein the instructions are to cause the processor to: apply a first weight to the second impactibility scores; apply a second weight to the third impactibility scores; and calculate the first impactibility scores based on the first weight applied to second impactibility scores and the third weight applied to the third impactibility scores, the first weight related to the second weight based on the first ratio.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment of a system for managing the allocation and cost of healthcare resources;

FIG. 2 illustrates an embodiment of a method for managing the allocation and cost of healthcare resources;

FIG. 3 illustrates examples of outcome perspectives;

FIG. 4 illustrates an embodiment for calculating impactibility scores;

FIG. 5 illustrates examples of values and scores that may be calculated in accordance with one or more embodiments;

FIG. 6 illustrates a graphical representation of impactibility scores calculated based on the example values and scores in FIG. 5; and

FIG. 7 illustrates an embodiment of a processing system for managing the allocation and cost of healthcare resources.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable.

In accordance with one or more embodiments, a system and method are provided to improve the allocation of healthcare resources to patients at a reduced cost to healthcare organizations, which are involved in providing or financing those resources. This may be accomplished using a dynamic approach for determining the location(s) where healthcare resources (e.g., physicians, clinical staff, services, equipment, etc.) will be offered and the specific timeframes for providing those services. This approach is especially beneficial for patients in areas that are economically disadvantaged or in remote areas where comprehensive healthcare is not available or offered on a regular basis.

In one embodiment, the facility- and office-based healthcare delivery system of an organization may be supported with the additional integration of existing provider specialties and/or the allocation of selected provider specialties. In these or other embodiments, a method and system are provided to improve the quality and financial outcomes of a healthcare organization (HCO) via access to care improvements, for example, through allocation or network integration of additional primary care physicians (PCPs) or specialists. Such a method and system, therefore, takes a health-economic approach to decision-making and may provide the opportunity to identify aspects where the HCO may expand its network. An HCO may be a medical insurance company, hospital, clinic, doctor's office, prescription services company, or another entity that provides or finances the cost of healthcare resources in a given geographical area.

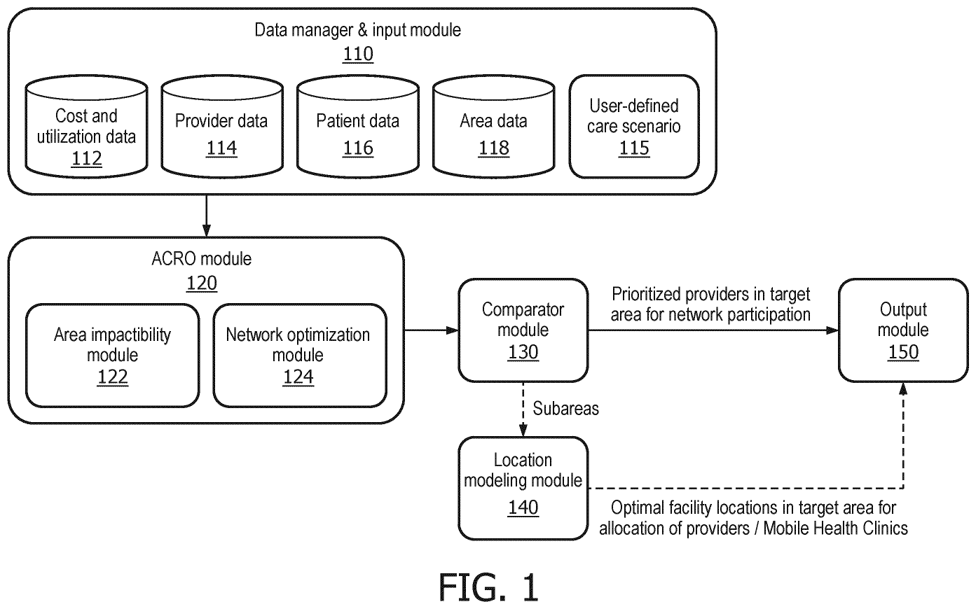

FIG. 1 illustrates an embodiment of a system for managing the allocation and cost of healthcare resources. The system performs computer modeling, based on complex algorithms and optimization techniques, to generate decisions for the purpose of allocating healthcare resources and costs in a given geographic area. The algorithms of the model may produce different decisions for one or more of outcome perspectives, thus suiting the interests of a particular HCO, its patients, or both.

Referring to FIG. 1, the system includes a data manager and input module 110, an Avoidable Cost Reduction Optimization (ACRO) module 120, a comparator module 130, a location modeling module 140, and an output module 150.

The data manager and input module 110 includes a plurality of storage areas, which, for example, may be included in a central database or which may be stored in different databases or storage locations (private and/or public) of the same network or a plurality of connected networks. Storage area 112 stores cost and utilization data, e.g., cost of providing various types of healthcare services, how often those services are required for a given population, cost of providing healthcare equipment, and other expenditures of the HCO. Storage area 114 stores healthcare provider data, e.g., affiliations, taxonomies, specialties and location, (preventive/RPM) service catalogue, cost and utilization data (e.g. healthcare insurance claims). Storage area 116 stores patient data, e.g., patient demographics and social determinants of health-related data. Storage area 118 stores area data, e.g., information indicating available transportation, road infrastructure, and/or other attributes of or relating to a catchment area.

The data manager and input module 110 may also store information in an area 115 that corresponds to one or more user-defined care scenarios. This information may be received from a user through the input module and may include inclusion and/or exclusion criteria defined by the user for an eligible patient cohort based on location and disease prevalence and comorbidities and sociodemographic criteria. In one embodiment, a care scenario may include additional information on which provider types (specialties) or (preventive) services should be considered within the Area Impactibility Module discussed below.

The ACRO module 120 includes the aforementioned area impactibility module 122 and a network optimization module 124. The area impactibility module 122 generates a score for one or more subareas in a catchment area of the HCO. The scores may be generated for a number of predetermined outcome perspectives selected based on certain information. The information may be pre-programmed to generate scores for subareas of the catchment area based on a particular health-cost approach to be taken. For example, the outcome perspective(s) may correspond to different health-economic scenarios, which scenarios may place greater emphasis (or weight) on allocating healthcare resources, healthcare costs, or a combination thereof. In one embodiment, the area impactibility module 122 may generate scores based on a potential reduction in avoidable costs compared with benefits to be realized by providing improved access to care or an extended delivery of selected services in one or more subareas. The area impactibility module 122 may calculate scores using one or more scoring functions that have, as inputs, information derived from the data manager and input module 110.

The network optimization module 124 may divide the catchment area (e.g., for an HCO) into a plurality of subareas and may cooperate with the area impactibility module to provide the computed scores for each of the subareas for comparison or analysis. An example of the operations performed by the network optimization module 124 are explained in greater detail below.

In one embodiment, the ACRO module 120 may risk-adjust potentially avoidable costs and event rates. The adjusted base cost or rates may be multiplied, for example, by an average risk score of a target population to determine an actual cost or rate. The ACRO module 120 may therefore solve the problem of identifying subareas in a catchment area that may benefit the most from increased access to specific care specialties, services, and/or programs. This may then lead to decreased healthcare expenditures in those subareas. At the same time, access to healthcare resources (e.g., the number of health units) may be increased for the population at risk in those areas. Through the particular scoring and optimization approach taken by the ACRO module 120, both the HCO and patients may benefit. For example, the health and outcomes of the targeted population may be improved, while simultaneously enhancing their experience. Also, per capita cost of care for the benefit of communities served may be reduced.

The comparator module 130 stores the output of the ACRO module 120 for one or more previous care scenario(s) and compares this output with one or more alternative care scenarios. For example, a previous care scenario may be defined for a congestive heart failure (CHF) base population with a set of (preventive) services provided by the selected specialty of cardiology. (In an alternative scenario, a user could modify the provider type to primary care or adjust the set of services, or the user may even select a different base population (e.g., diabetes) where services are rendered by endocrinologists.) The comparator module 130 may then rank the scenario(s) by impact on one or more selected outcome perspectives, and output information indicative of providers in a target subarea for network participation.

If no existing providers are available to enter into a participation agreement with the HCO for a target subarea, the location modeling module 140 may be used to determine one or more facility locations in the target subarea for allocating providers and/or mobile health clinics (MI-ICs). The location modeling module 140 may make this determination based on information corresponding to the subareas (e.g., prioritized subareas) output from the comparator module 130. In one embodiment, the location modeling module 140 may include a healthcare facility location (HFL) optimizer which determines one or more optimal facility locations based on the subarea information output from the comparator module 130.

In one embodiment, for each subarea with an assigned provider, the location modeling module 140 may determine the optimal location using an HFL algorithm. Models for location problems may be divided into four classes: analytic, continuous, network, and discrete. HFL problems may be discrete in nature and, for example, may be addressed by covering-based, median-based or other models. In such models, demands generally arise on the nodes (e.g. cities, patient homes) and facilities are restricted to a finite set of candidate locations, which may include the demand nodes. The algorithmic design of such models is known, and the output of the HFL optimizer may be a specific location to position the provider for each subarea.

The output module 150 receives the information indicative of the providers in the target subarea(s) from the comparator module 130. The providers may be prioritized by the comparator module 130 for network participation, and then healthcare resources (e.g., providers and/or MHCs) may be allocated in the target subarea(s) in view of the cost dictated by the selected outcome perspective. Additionally, or alternatively, the output module 150 may receive information from the location modeling module 140 indicative of one or more optimal facility locations in the target subarea(s). The output module 150 may then output this information so that providers and/or MHCs may be allocated to the target subarea(s).

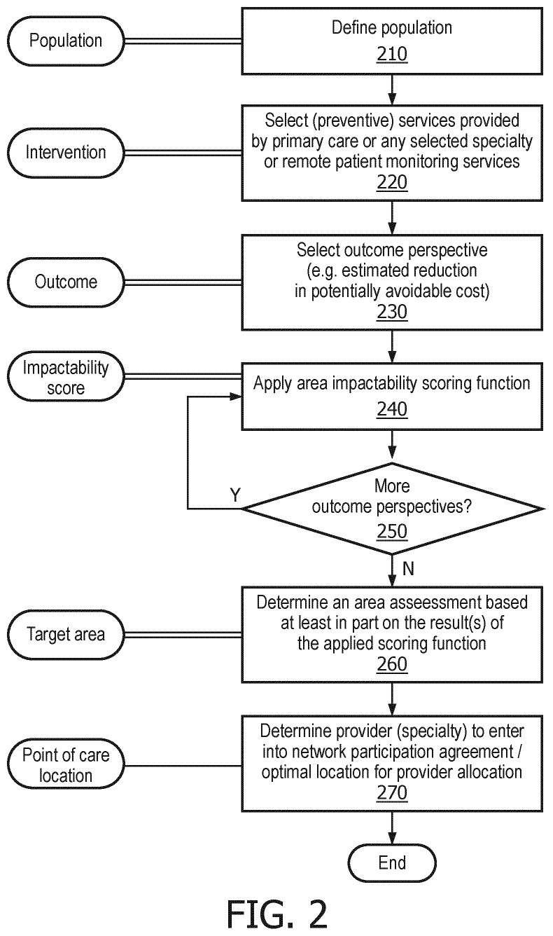

FIG. 2 illustrates an embodiment of a method for managing the allocation and cost of healthcare resources for a plurality of outcome perspectives. The method may be, partially or wholly, performed by the modules of the system of FIG. 1. In one embodiment, the method may perform an allocation that improves the quality of or access to healthcare services in a target subarea in the most cost-effective manner possible to the healthcare organization. In another embodiment, the method may be implemented in a manner that favors reduced costs over healthcare access in the target subarea, or vice versa.

An initial operation 210 includes defining a patient population. The patient population may be defined based on one or more predetermined criteria. In one embodiment, the predetermined criteria include a disease inclusion criteria and specific exclusion criteria.

The disease inclusion criteria may define what diseases are to be taken into consideration and what diseases may be excluded in the decision-making process. For example, the disease inclusion criteria may involve a first set of diseases which are expected to commonly occur or occur under a given set of circumstances (the flu at certain times of the year, diabetes, heart disease, etc.). The disease inclusion criteria may also take into consideration certain ethnic classes which have a propensity to develop certain diseases relative to other ethnic classes, geographical location exposed to certain specific pathogens or other health conditions, and/or environmental concerns where certain types of diseases are especially prevalent. The specific exclusion criteria may involve a second set of diseases which are not expected to commonly occur and/or are ones for which treatment is difficult to provide remotely, for example, because of the need for special equipment, doctors, etc.

In operation 220, an intervention for a patient cohort is selected based on one or more specific predetermined (e.g., preventive) services offered by a primary care physician (PCP) or provider of selected specialty. In one embodiment, Remote Patient Monitoring (RPM) technology may be used to prioritize patients within a given subarea. RPM technology allows one or more physiologic patient parameter(s) to be collected, stored, and transmitted, for example, to physicians, clinical staff, or other healthcare professionals. Examples include, but are not limited to, electrocardiogram (ECG), glucose monitoring, weight, blood pressure, pulse oximetry, respiratory flow rate, and/or other parameters.

When RPM technology is used, physiologic monitoring and treatment management services may be sent back to the patient or caregiver (at the patient site) based on an interpretation of the recorded and transmitted data by the physicians, clinical staff, or other healthcare professionals. These professionals may be located, for example, at a hospital, health center, or other main healthcare facility. In one embodiment, patient prioritization for RPM services may be performed, for example, based on one or more assessed acuity levels, (chronic/other) conditions, healthcare utilization history in a past time period (e.g., the previous 12 months, including the number of ED visits, hospitalizations, and other forms of care), resident status, and/or other patient demographics.

In operation 230, one or more outcome perspectives are selected. The outcome perspectives may address, for example, an estimated reduction in potentially avoidable costs, a gain potential in saved health units, and/or potential in cost-saving effectiveness where the potential may be impacted the most (or in a certain way) by improved access to selected (preventive) services within a selected time period.

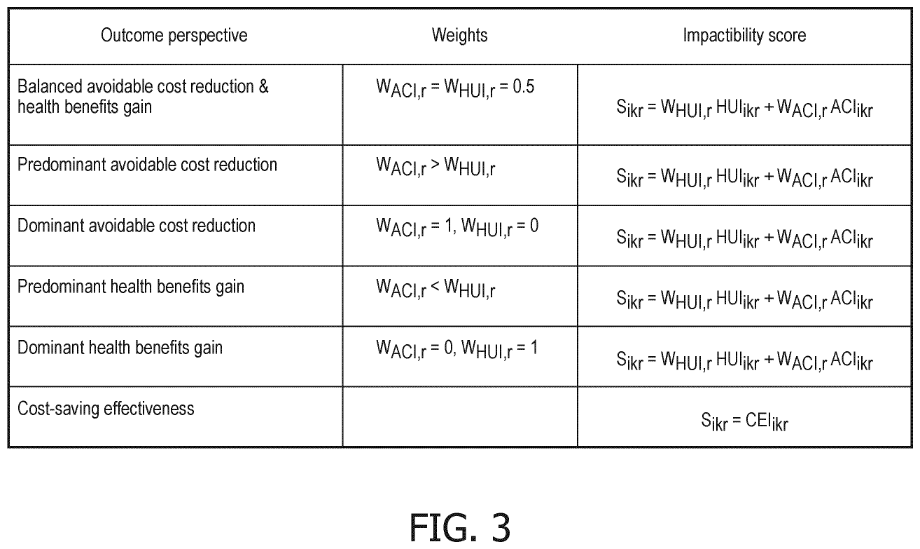

FIG. 3 illustrates examples of outcome perspectives available for selection in operation 230. The outcome perspectives include various ways of allocating HCO costs with access to healthcare resources. The perspectives include (i) balanced avoidable cost reduction & health benefit gain, (ii) predominant avoidable cost reduction, (iii) dominant avoidable cost reduction, (iv) predominant health benefit gain, (v) dominant health benefit gain or (vi) cost-saving effectiveness. As discussed in greater detail below, one or more of the outcome perspectives may be realized based on predetermined ratio of healthcare resources and cost, which, for example, may be determined by weight values applied in one or more associated impactibility score functions.

Referring to FIG. 3, some of the outcome perspectives take into consideration avoidable costs. In one embodiment, the avoidable costs may be linked to a defined patient cohort living in one or more subareas of the catchment area of an HCO. The avoidable costs may be determined, for example, based on claims data stored in area 112 of the data manager & input module 110. In one embodiment, observed avoidable cost may be determined retrospectively, for example, by summing the claim amounts for services provided to treat potentially avoidable health care events occurring in a defined time period. Such information may also be stored in module 110. An estimated avoidable cost for a defined prospective time period may be derived based on a prediction of historical, previously observed events and associated risk factors, including but not limited to determinants of health associated with social activity. This information may also be obtained from module 110. (In accordance with at least one embodiment, the terms “avoidable cost” or “preventable cost” may be used interchangeably with observed or estimated avoidable cost).

In one embodiment, there may be two types of preventable costs contributing to a total avoidable episodic cost: (i) costs related to complications of a disease or condition itself and (ii) costs related to complications of patient safety incidents. For each episode of care (Dₖ), potentially avoidable events may be categorized into categories me or complication classifications, e.g., hypotension or fluid and electrolyte disturbances in patients with chronic heart failure. For each classification, it may be assumed that a set of (preventive) services (R_{f}) is available that may potentially prevent the occurrence.

In accordance with one embodiment, a clinically preventable cost (CPC) may be defined for a selected condition or episode of care (Dₖ) over a time period Δt. In this case, the CPC may be the amount of total episodic cost which can be potentially prevented, given access to timely services and installation of preventive services R_{f} for each condition Dₖ in an area Aᵢ.

In one embodiment of operation 230, the stationary facility- and office-based healthcare delivery system of the HCO may be supported with mobile health clinics. An MHC may essentially be thought of as a physician's office and clinic on wheels. For example, an MHC may be a specially outfitted truck that includes examination rooms, laboratory services, medical tests, and/or other professional services provided by physicians or nurses. MHCs can move to remote areas where patients have little or no access to medical facilities. MHCs can also directly visit patients who do not have the resources to travel to obtain care.

MHCs may therefore be flexibly tailored to meet the healthcare needs of target populations in selected areas or when no providers of a needed specialty are available for network integration. In addition, mobile units may deliver high-impact care to hard-to-reach, vulnerable populations and individuals with chronic diseases in a cost-effective manner. MHCs may then serve as a platform to help patients to (re)connect with medical and/or social resources in their communities, which may eventually lower healthcare expenses for avoidable events. In view of these considerations, MHCs may enable avoidable cost savings and improve health outcomes, for example, in underprivileged or economically disadvantages subareas in the catchment area.

Referring again to FIG. 2, in operation 240, areas in the catchment area of the HCO are scored in respect to one of the outcome perspectives selected, for example, to implement a certain health-economic approach. The scoring may be performed by the area impactibility module 122 in FIG. 1. The resulting score may be referred to as an impactibility score and may be calculated based on a predetermined scoring function. In one embodiment, each of the subareas in the catchment area may be scored based on a potential reduction in avoidable costs versus (or weighed against) improved access to care or the extended delivery of selected services in that subarea.

In one embodiment, once the subareas are scored, the scores may be used as inputs to a healthcare facility location solution (e.g., determined by comparator module 130 and/or location modeling module 140) to determine the exact subarea(s) or locations in the subarea(s) where healthcare resources are to be allocated and the type, number, and/or nature of those resources for a given cost scenario. At that point, a complete plan may be developed (and output through output module 150) for a healthcare organization (HCO) to follow in order to solve the problem of reducing or minimizing avoidable costs in a flexible manner.

In operation 250, impactibility scores may be calculated, by the area impactibility module 122, for one or more additional outcome perspectives. The additional outcome perspectives may be automatically selected based on pre-programmed instructions (e.g., in one embodiment, scores may be automatically calculated for all or a portion of the outcome perspectives), and the scores may then be compared by the comparator module 130. The subarea(s) having the greatest score may then, for example, be selected for an allocation of costs and healthcare resources for the corresponding selected outcome perspective.

In operation 260, an area assessment may be determined based, at least in part, on the calculated impactibility score(s). This may involve identifying one or more target areas which, given resource constraints of the organization, are expected to have the highest impact on the selected outcome perspective.

In operation 270, a determination is made as to the provider(s) who will enter into a network participation agreement and/or the location where the provider(s) will be allocated. The provider(s) may be general-care physician(s), general-care clinical staff member(s), specialized physician(s), specialized clinical staff member(s), and/or other type of healthcare professionals or HCO personnel. The determined location may be, for example, an optimal location given the subarea assessment performed in operation 180.

In one embodiment, the actual point of care of the provider(s) may be identified within one or more prioritized target subareas. If a specialized healthcare professional is required and there are already one or more providers of the selected specialty in the target subarea(s), then those one or more providers may enter into a network participation agreement (e.g., contracted provider status), if possible. In one embodiment, the providers may be prioritized, for example, based on social network measures for provider patient-sharing networks.

In one embodiment, providers and/or MHCs may be allocated to one or more target subareas on temporarily basis. The optimal point-of-care location (e.g., office address) for provider allocation in the target subarea(s) may be determined by one or more predetermined models, for example, based on location theory. An example of such a model is disclosed in “A Survey of Healthcare Facility Location,” Computers & Operations Research 79 (2017), pp. 223-263, by Ahmadi-Javid et al. (The purpose of the model disclosed in this article is to identify what problems are in a given area, not to determine an optimal allocation of healthcare resources and the costs associated with providing those resources for a given subarea of a catchment area for one or more selected outcome perspectives, as is the case with one or more embodiments described herein.)

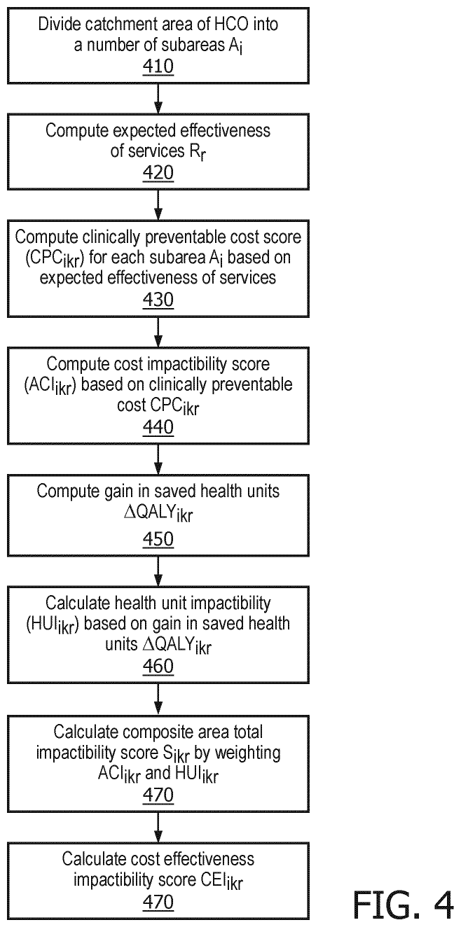

FIG. 4 illustrates an embodiment of operations which may be performed by the ACRO module 120 in FIG. 1. In operation 410, the catchment area of the HCO is divided into a number of subareas Aᵢ, where i is indexed from 1 to I which are integers. The catchment area is an area of service for the HCO and may be divided, for example, based on existing zip code (ZIP) areas, census tracts, counties, or other geographically defined areas.

Once subareas A have been determined, the avoidable cost impactibility (ACIᵢₖ) score, the health unit impactibility (HUIᵢₖᵣ) score, and the cost effectiveness impactibility (CEIᵢₖᵣ) score may be calculated by the area impactibility module 122 for each subarea Aᵢ. One or more of these scores may also be calculated based on a condition Dₖ, where k=1 . . . K, that may be determined to cause avoidable costs in the network, for example, based on analytics, information in module 110, and/or other information. In one embodiment, Dₖ may also refer to an episode—delivering all services to treat a particular diagnosis (condition) within a time period. The impactibility scores for each subarea may indicate, for example, how well the subarea would respond with respect to increased access to care and delivery of services Rᵣ, where r=1 . . . R for condition Dₖ. Examples for computing the impactibilty scores will now be discussed.

In operation 420, the expected effectiveness of the services Rᵣ (% Effectiveness) may be estimated based on Equation (1):

% Effectiveness=% initial or expected patient adherence to newly network-participating/allocated provider in Δt×% adherence with follow-up at newly network participating/allocated or existing accountable provider×% effectiveness of treatment in reducing potentially avoidable events (1)

In one embodiment, a value indicative of patient adherence to treatment services may be estimated based on standard literature data for a selected treatment Area. In one embodiment, local population specific-adherence may be measured based on a delivery time interval. Data updates may be stored in the data manger and input module 120 and subsequently used to refine the score calculation performed by the area impactibility module 122.

In operation 430, a condition-specific actually observed or estimated clinically preventable cost CPCᵢₖᵣ for a target population served in each subarea Aᵢ may be calculated in Equation (2) based on the expected effectiveness of services Rᵣ (% Effectiveness) and information output from the data manager and input module 110.

CPCᵢₖᵣ=risk-standardized avoidable episodic event cost in area×average risk average risk score of target population×observed or estimated potentially preventable event rate×target population size in subarea×% expected effectiveness of the service r (2)

In operation 440, the cost impactibility ACLᵢₖᵣ score is computed for each episode Dₖ, using the rescale function in Equation (3), based on the condition-specific actually observed or estimated clinically preventable cost CPCᵢₖᵣ in each subarea Aᵢ:

ACIᵢₖᵣ=rescale(CPCᵢₖᵣ−Σᵢ₌₁^{I}Cᵢₖ, to =(1,score_max)) (3)

In Equation (3), the observed amount of total potentially avoidable costs Cᵢₖ for an episode Dₖ incurred by patients living in subarea Aᵢ may be computed based on cost and utilization data 112 in the data manager and input module 112 in FIG. 1. The rescale function maps the range of the cost differential to values between 1 and a predetermined score (score_max).

In operation 450, the gain in saved health units Δ [symbol] ALYᵢₖᵣ, for each set of services Rᵣ, may be estimated based on Equation (4) for each condition Dₖ in each subarea Aᵢ. The gain in saved health units Δ [symbol] ALYᵢₖᵣ may be measured, for example, in quality-adjusted life years (QALYs).

ΔQALYᵢₖᵣ=QALYᵣ×observed or estimated potentially preventable event rate×target population size in area×% expected effectiveness of the service r (4)

In Equation (4), [symbol] ALYᵣ refers to the average number of health units which can be saved by providing service r to a population with condition Dₖ at risk for potentially preventable events.

In operation 460, a health unit impactibility HUIᵢₖᵣ score for an episode Dₖ for each subarea Aᵢ is calculated using the rescale function in Equation (5). As shown, the rescale function is calculated based on the gain in saved health units Δ [symbol] ALYᵢₖᵣ calculated in Equation (4).

HUIᵢₖᵣ=rescale(ΔQALYᵢₖᵣ, to =(1,score_max)) (5)

In one embodiment, both impactibility scores ACIᵢₖᵣ and HUIᵢₖᵣ may be scaled to the same range, e.g., (1, score_max), to ensure comparability and aggregation. Also, these impactibility scores may be constructed in a way that an increase in the score value reflects an increase of impactibility on avoidable cost reduction in respective gain of health units.

In operation 470, a composite area total impactibility score Sᵢₖᵣ may be computed, for example, based on a predetermined ratio of healthcare resources (e.g., allocation of heath units) to cost (e.g., for providing the health units) in one or more subareas based on the selected outcome perspective. The predetermined ratio may include a ratio of weights w_{HUI,r} and w_{ACI,r} applied to the cost impactibility ACIᵢₖᵣ and gain in saved health units Δ [symbol] ALYᵢₖᵣ scores, respectively, by Equation (6).

Sᵢₖᵣ=w_{HUI,r}HUIᵢₖᵣ+w_{ACI,r}ACIᵢₖᵣ (6)

where the weights are set so that w_{HUI,r}+w_{ACI,r}=1.

In one embodiment, if only cost prevention is of concern, the weights w_{HUI,r} may be set to zero. Similarly, if v_{ACI,r}=0, the gain in health units is the measure of interest for the subareas A. Otherwise, a weighted combination of impact on cost and health gain may be used to provide the S score for the subareas given the selected outcome perspective.

In operation 480, the cost effectiveness impactibility score CEIᵢₖᵣ may be computed based on the rescale function in Equation (8), where CERᵢₖᵣ corresponds to an area cost effectiveness ratio.

CEIᵢₖᵣ=rescale(CERᵢₖᵣ, to =(1,score_max)) (7)

The area cost effectiveness ratio CERᵢₖᵣ may be defined based on the cost per QALY saved, as indicated in Equation (8).

$${CER}_{ikr} = \frac{service cost spent - {CPC}_{ikr}}{Δ {QALY}_{ikr}} \qquad ( 8 )$$

In some cases, an equation consisting of the ratio of two differences (e.g., like Equation (8)) might not be available for use. For instance, when there is no or very little QALY improvement, the denominator in Equation (8), e.g., /delta QALY, may be zero or nearly Zero. In addition, if this ratio is negative, it might be because of a cost difference being negative or the QALY being negative. In these cases, a different equation may be used to calculate CERᵢₖᵣ.

As previously indicated, the HCO may choose among different outcome perspectives, as shown in FIG. 3, for purposes of computing impactibility scores. The outcome perspectives include balanced avoidable cost reduction & heath benefits gain. In this case, the weights w_{HUI,r} and w_{ACI,r} for computing the composite area total impactibility score Sₖ may be the same value equal to 0.5. When the outcome perspective is predominant avoidable cost reduction, w_{ACI,r} may be greater than w_{HUI,r}. When the outcome perspective is dominant avoidable cost reduction, w_{HUI,r} may equal zero and w_{ACI,r} may equal 1. When the outcome perspective is predominant heath benefits gain, w_{ACI,r} may be less than w_{HUI,r}. When the outcome perspective is dominant heath benefits gain, w_{HUI,r} may be equal to 1 and w_{ACI,r} may be equal to zero. In all of these cases, the composite area total impactibility score Sᵢₖᵣ may be calculated using Equation (6).

When the composite area total impactibility score Sᵢₖᵣ is to be calculated for an outcome perspective corresponding to cost-saving effectiveness, score Sᵢₖᵣ may be calculated by Equation (9).

Sᵢₖᵣ=CEIᵢₖᵣ (9)

where the cost effectiveness impactibility score CEIᵢₖᵣ is calculated by Equation (7).

The subareas Aᵢ into which a provider may enter into a network participation agreement or to which a provider or other HCO participant may be allocated may be performed by the network optimization module 124 of the ACRO module 120. In one embodiment, the outputs of the network optimization module 124 may be given as a binary result RAᵢᵣ (0 or 1) for each subarea Aᵢ, where RAᵢᵣ=1 indicates the delivery of service r offered by an allocated or to-be-contracted provider in subarea Aᵢ. To identify the subareas Aᵢ into which a provider can enter for a network participation agreement or into which the provider may be allocated, one of the following two objective functions (10) or (11) may be solved depending which outcome measure or perspective was selected:

maximize Σᵢ₌₁^{I}Σₖ₌₁^{K}Σᵣ₌₁^{R}Sᵢₖᵣ·RAᵢᵣ with RAᵢ∈{0,1} (10)

maximize Σᵢ₌₁^{I}Σₖ₌₁^{K}Σᵣ₌₁^{R}CEIᵢₖᵣ·RAᵢᵣ, with RAᵢ∈{0,1} (11)

Equations (10) and (11) may be applied given the following constraints:
1. Service delivery sites cannot exceed the total number of providers available of a selected specialty. This constraint may be expressed by Equation (12), where NUMProvider corresponds to the total number of available providers for a selected specialty.

0≤Σᵢ₌₁^{K}(ifelse(Σᵣ₌₁^{R}RAᵢᵣ≥1;1;0))≤NUMProvider (12)
2. The total program delivery cost (TPDC) must be less than or equal to the available program budget. This constraint may be expressed by Equation (13).

TPDC=system, operational and FTE cost+Expected service delivery in period Δt×average total cost per service (13)

Based on Equations (10) to (13), the network optimization module 124 may output a solution for a given outcome perspective. In one embodiment, the solution may maximize the selected impactibility score and at least in this sense may be considered optimal. One or more known integer programming techniques may be used, for example, to compute the solution.

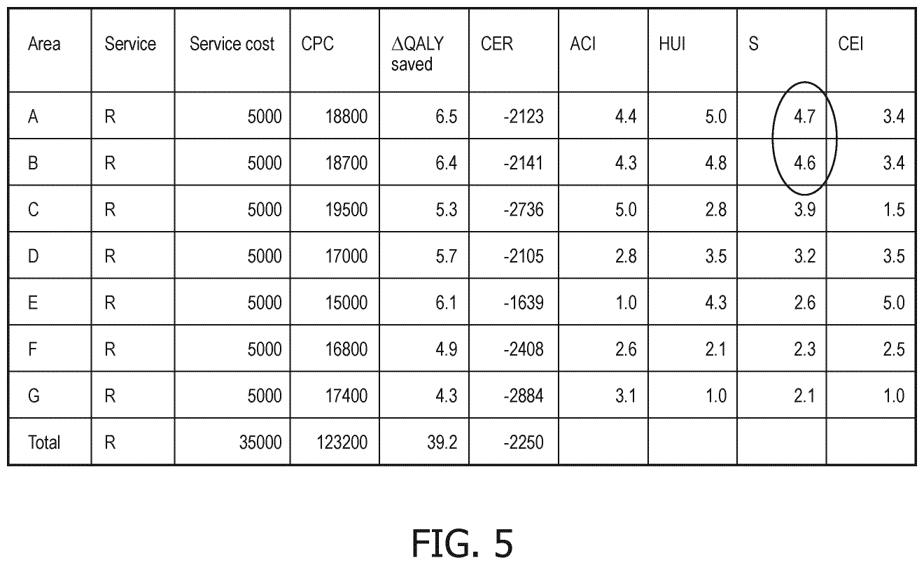

FIG. 5 illustrates values calculated for subareas A (in this example, seven values listed A to G) of a catchment area according to one example embodiment. In FIG. 5, the values were computed using Equations (1) to (9) for the same service R and the same service cost and for the balanced cost reduction & health benefit outcome perspective. The values for Equations (1) to (9) were generated based on information in the storage areas and a user-defined scenario of the data manager & input module 110 corresponding to case studies.

In FIG. 5, values were generated for clinically preventable cost (CPC), gain in saved health units (ΔQALY), and cost effectiveness ratio (CER). In addition, four impactibility scores were calculated: area cost impactibility (ACI) score, health unit impactibility (HUI) score, composite area total impactibility (S) score, and cost effectiveness impactibility (CEI) score. The CPC value was the greatest for subarea C. The Δ [symbol] ALY value was the greatest for subarea A. The CER value had the greatest (negative) value for subarea G. The ACI score was the greatest for subarea C. The HUI score was the greatest for subarea A. The S score was the greatest for subarea A and the second greatest S score was for subarea B, as indicated by the oval. The CEI value was the greatest for subarea E.

These scores may be input from the ACRO module 124 to the comparator module 130, which may compare the values and scores and select one or more of the subareas that satisfy the selected outcome perspective, which, in this case, is the balanced cost reduction & health benefit outcome perspective. In one embodiment, the comparator module 130 may designate the subareas with the greatest composite area total impactibility score S for purposes of allocating resources, e.g., a provider to be allocated to those subareas and/or to enter into a network participation agreement.

In this case, the comparator module 130 may designate that subareas A and B for output to the output module 150, because the S scores for subareas A and B have the greatest values (4.7 and 4.6, respectively) relative to the other subareas. In one embodiment, the S scores may represent a weighted sum of the health unit impactibility (HUI) score and the area cost impactibility (ACI) score, e.g., an indication of providing additional health services or resources in a given subarea versus the cost of providing those services or resources in that subarea. The resulting S score may be, for example, a weighted average (or some other function) of the HUI and ACI scores. The subarea(s) having the highest S score may therefore represent the subareas that have the most balanced solution to providing healthcare services or resources (e.g., heath units) given the cost considerations.

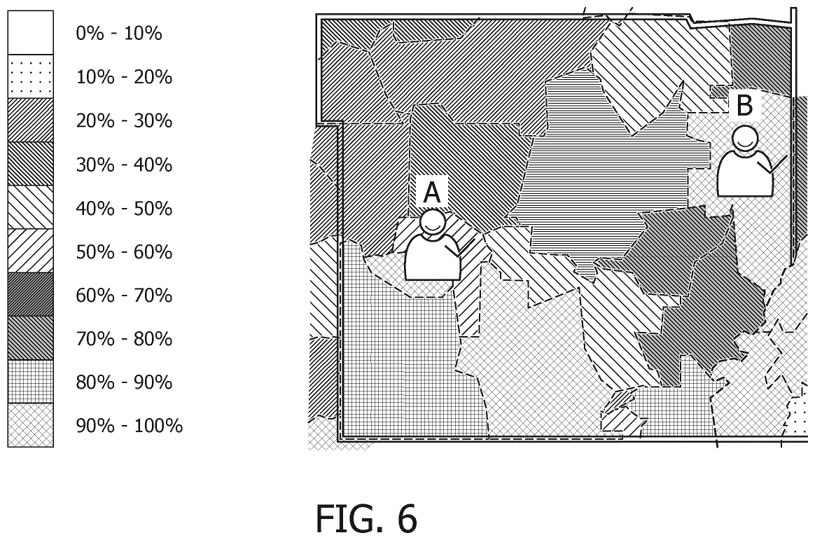

FIG. 6 illustrates an example of a graphical representation of the impactibility scores S corresponding to FIG. 5. In FIG. 6, the impactibilty scores are calculated for a catchment area divided into subareas A to G, which, in this case, correspond to ZIP code areas, as output from the network optimization module 124. The impactibility scores fall into ranges that correspond to different degrees of shading. The lighter shades represent lower impactibility scores and the darker shades higher impactibility scores. As indicated by the scores in FIG. 6, subareas A and B have the greatest S scores and thus are the subareas with the darkest shading. Based on these S scores (and given the selected outcome perspective) it may be determined that two providers would potentially sign an agreement for network participation in subareas A and B, respectively, or that two providers would be respectively allocated to those areas to provide healthcare services.

If there is a limit on how many providers should be contracted in a subarea or the catchment area, the providers may be prioritized, for example, using social network measures for provider patient-sharing networks. In one embodiment, providers may be prioritized based on one or more of the following network measures, which may be derived from insurance claims data and/or other data, including but not limited to the data stored in the data manager & input module 110.
Number of shared patients between a focal provider and other in-network providers
Centrality or specialty-specific relative centrality, e.g., measuring the centrality of a focal provider type (e.g. Cardiology) relative to that of another specialty (e.g., Internal Medicine)
Degree which counts the number of in-network providers with whom the focal providers shared patients, or an adjusted degree which divides the degree by the number of shared patients.

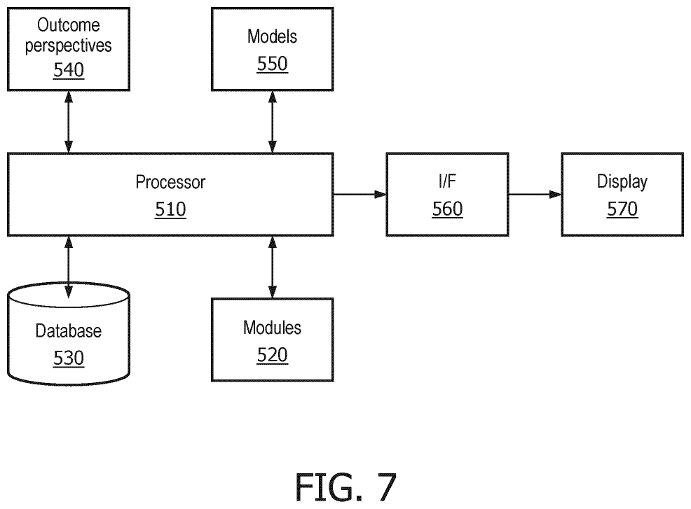

FIG. 7 illustrates an embodiment of a processing system 500 for managing the allocation and cost of healthcare resources. The processing system may include the modules and other features of FIG. 1 or may include one or more feature different from the system of FIG. 1.

Referring to FIG. 7, the processing system 500 includes a processor 510, a machine-readable storage medium 520, a database 530, storage areas 540 and 550, an interface 560, and a display 570. The processor 510 may be implemented in logic which, for example, may include hardware, software, or both. When implemented at least partially in hardware, the processor 510 may be, for example, any one of a variety of integrated circuits including but not limited to an application-specific integrated circuit, a field-programmable gate array, a central processing unit, a combination of logic gates, a system-on-chip, a microprocessor, or another type of processing or control circuit.

When implemented in at least partially in software, the processor 510 may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the operations and methods of the embodiments described herein.

The machine-readable storage medium 520 stores instructions for controlling some of the operations of the processor 510. In one embodiment, the instructions control the processor 510 to perform the operations of the method and system embodiments described herein, including but not limited to the operations of the modules illustrated in FIG. 1. In this case, the modules may be implemented in any of the forms of logic (software, hardware, or a combination) described herein. For illustrative purposes, the instructions in storage medium 520 are labeled as including modules.

The database 530 stores various information that may be used by processor 510 to perform one or more of the aforementioned operations. In one embodiment, the database 530 may store the data in the data manager & input module 110 in FIG. 1. The database 530 may also store the user-defined care scenario or information corresponding to this scenario may be directly input by a user. Additionally, the database may be or include a centralized database, a decentralized database (e.g., blockchain), or a storage network of databases respectively storing patient data, insurance claim data, area data, cost/utilization data, and/or other information. In one embodiment, the database 530 may be at least partially implemented in a cloud-based network.

The outcome perspectives 540 and models 550 may be stored in different storage areas coupled to the processor 510 or may be stored in database 530. The outcome perspectives and models (e.g., location modeling) may be the ones described in connection with previous embodiments.

The interface 560 may be implemented in hardware, software, or both. When implemented in hardware, the interface 560 may include a port, connector, pin configuration, cable, or signal lines. In one embodiment, the interface may include a wireless interface (e.g., WiFi, GSM, CDMA, LTE, or other mobile network), or an interface compatible with another type of communication protocol). In this latter case, the processing system may be located remotely from the display 570, e.g., may be included in a virtual private network accessible by personnel at different locations. When implemented in software, the interface 560 may be, for example, application programming interface (API) running on a workstation, server, client, or mobile device.

In operation, the instructions stored in the machine-readable medium 520 controls the processor 510 to perform the operations of the method and system embodiments described herein. The processor may receive inputs from one or more users, applications, and/or control software during this time to control, change, or implement some of these operations. The results of the processor 510, including a designation of an allocation of healthcare resources and cost for one or more subareas of an HCO catchment area, may be output to the display 570 for one or more selected outcome perspectives.

### Technological Innovation

One or more embodiments described herein address a problem and/or provide a technical solution to allocating healthcare resources in a way not previously known or practiced. For example, one problem in the field is the inability to effectively resolve the trade-off between the number and type of healthcare resources to be allocated in a geographical area and the costs associated with allocating those resources. Existing approaches allocate healthcare resources on the basis of the opinions and judgment of healthcare personnel. These personnel are unable to make informed decisions as to the best way to allocate care versus cost, at least in a way that proves to be beneficial for both healthcare organizations and the patients they serve. As a result, the organizations fail to deliver the best care needed by patients and, at the same time, make wasteful expenditures. Often times, healthcare personnel are not even aware that a specific type of care is lacking in a certain area.

One or more embodiments described herein solve this problem by performing a complex analysis for managing the allocation and cost of healthcare resources in a specific way to achieve a specific purpose. This analysis may be performed on a geographical basis, which may include rural areas where healthcare services are not readily available or in underprivileged or economically depressed areas. The embodiments therefore provide a solution which is not merely abstract in nature.

In at least one embodiment, areas were critical healthcare needs are not being met are identified using statistical and analytical methods and then one or more values are computed to represent an allocation of resources that satisfy the needs in a cost-effective manner possible. In these or other embodiments, different outcome perspectives are analyzed and preferences considered toward generating qualitative information that may be used as a basis for delivering the best care to geographical areas for given cost objectives.

In at least one embodiment, multiple solutions are generated for different outcome perspectives and a result is generated that best suits the differing interests of healthcare organizations, while at the same time meeting the needs of their patients. The embodiments described herein, therefore, provide significantly more than merely the idea of merely allocating healthcare resources. At the very least, the embodiments represent a practical application to healthcare resource allocation versus cost that provide real-world beneficial results. The embodiments are especially beneficial for patients in areas that are economically disadvantaged or in remote areas where comprehensive healthcare is not available or offered on a regular basis.

Additionally, while one or more features of the embodiments may involve the use of a mathematical formula, the embodiments are in no way restricted solely to a mathematical formula. Nor are they directed to a method of organizing human activity or a mental process. Rather, the complex and specific approach taken by the embodiments, combined with the amount of information processing performed, negate the possibility of the embodiments being performed by human activity or a mental process. Moreover, while a computer or other form of processor may be used to implement one or more features of the embodiments, the embodiments are not solely directed to using a computer as a tool to otherwise perform a process that was previously performed manually.

Nor do these embodiments preempt the general concept of allocating healthcare resources. For example, since the inception of private healthcare insurance, healthcare organizations have allocated budgets to servicing patients. The embodiments described herein do not preempt, or otherwise restrict the public from practicing the general concept of allocating healthcare resources. Rather, the embodiments take a specific approach (e.g., through the generation and comparison of impactibility scores, subarea assessment, and/or other features) to achieve a specific purpose, e.g., a healthcare allocation solution customized to satisfy the differing interests of healthcare insurers, providers, and/or other health-related organizations. This may improve insight on services needed for patients to be targeted by a defined program and the likely impactable cost by such an intervention.

Because the embodiments take a specific and unique approach to allocating healthcare resources in view of cost or other considerations, it is further noted that the embodiments do not cover activity considered to be merely well-understood, routine, and conventional. Rather, as previously discussed, the embodiments overcome problems that really never have been adequately solved in the healthcare industry. Moreover, the embodiments improve the functioning of a processor or computer when used to implement one or more operations described herein, at least when it comes to weighting, processing, and comparing the allocation of resources and cost in view of outcome perspectives on a subarea basis.

Moreover, in accordance with one or more of the embodiments, extended access to care to selected physician types may be provided by providing services with the highest impact on reduction of avoidable cost of potentially preventable events or a gain of saved health units in that area. Additionally, providers of a selected specialty or primary care physicians may be prioritized in such target areas for entering into a network participation agreement. The term provider may also refer to physician, qualified healthcare professional, or clinical staff. Clinical staff includes, for example, RNs and medical assistants. In addition, one or more embodiments described herein may achieve the triple aim of improving the health of a targeted population, while at the same time enhancing experience and patient outcome and reducing the per capita cost of care for the benefit of communities served.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein. In one or more embodiments, the operations performed by the method and system may be implemented, at least partially, using logarithmic arithmetic. This approach may be beneficial for performing complex operations that require many (fixed-point) floating point operations, in addition, subtraction, multiplication and division. The use of logarithmic arithmetic for these purposes may therefore improve speed and accuracy.

The modules, models, managers, and other information processing, calculating, and computing features of the embodiments disclosed herein may be implemented in logic which, for example, may include hardware, software, or both. When implemented at least partially in hardware, the modules, models, managers, and other information processing, calculating, and computing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the modules, models, managers, and other information processing, calculating, and computing features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method for managing healthcare resources, comprising:
receiving information selecting a first outcome perspective;
calculating first impactibility scores for the first outcome perspective;
determining a first subarea based on the first impactibility scores; and
designating an allocation of healthcare resources and cost for the first subarea based on the first outcome perspective, wherein the first impactibility scores are calculated for respective subareas including the first subarea and wherein the first outcome perspective corresponds to a first ratio of healthcare resources and cost.

2. The method of claim 1, wherein generating the first impactibility scores includes:
calculating second impactibility scores for respective ones of the subareas;
calculating third impactibility scores for respective ones of the subareas; and
calculating the first impactibility scores based on the second impactibility scores and the third impactibility scores.

3. The method of claim 2, further comprising:
applying a first weight to the second impactibility scores;
applying a second weight to the third impactibility scores; and
calculating the first impactibility scores based on the first weight applied to second impactibility scores and the third weight applied to the third impactibility scores, the first weight related to the second weight based on the first ratio.

4. The method of claim 2, further comprising:
calculating each of the second impactibility scores based on a gain in saved health units for a respective one of the subareas and for a number of episodes; and
calculating each of the third impactibility scores based on condition-specific actually observed or estimated clinically preventable cost for a respective one of the subareas and for the number of episodes.

5. The method of claim 4, wherein at least one of the second impactibility scores and the second impactibility scores is calculated based on at least one condition, the at least one condition determined to cause avoidable costs in a provider network.

6. The method of claim 4, further comprising:
determining the condition-specific actually observed or estimated clinically preventable cost for a respective one of the subareas and for the number of episodes based on cost and utilization data.

7. The method of claim 1, wherein the first subarea is determined based on a greatest one of the first impactibility scores.

8. The method of claim 1, further comprising:
determining one or more potentially avoidable costs; and
designating the allocation of healthcare resources and costs based on a reduction of the one or more potentially avoidable costs.

9. The method of claim 1, further comprising:
receiving information selecting a second outcome perspective;
calculating the first impactibility scores for the second outcome perspective;
designating an allocation of healthcare resources and cost for the first subarea based on the second outcome perspective, wherein the first impactibility scores are calculated for the respective subareas including the first subarea and wherein the second outcome perspective corresponds to a second ratio of healthcare resources and cost different from the first ratio.

10. The method of claim 9, further comprising:
comparing the first impactibility scores generated for the first outcome perspective and the first impactibility scores generated for the second outcome perspective; and
selecting the first impactibility scores generated for the first outcome perspective.

11. A system for managing healthcare resources, comprising:
an interface; and
a processor configured to receive information selecting a first outcome perspective, calculate first impactibility scores for the first outcome perspective, determine a first subarea based on the first impactibility scores, and output information through the interface indicative of a designation of an allocation of healthcare resources and cost for the first subarea based on the first outcome perspective, wherein the processor is configured to calculate the first impactibility scores for respective subareas including the first subarea and wherein the first outcome perspective corresponds to a first ratio of healthcare resources and cost.

12. The system of claim 11, wherein processor is configured to:
calculate second impactibility scores for respective ones of the subareas;
calculate third impactibility scores for respective ones of the subareas; and
calculate the first impactibility scores based on the second impactibility scores and the third impactibility scores.

13. The system of claim 12, wherein the processor is configured to:
apply a first weight to the second impactibility scores;
apply a second weight to the third impactibility scores; and
calculate the first impactibility scores based on the first weight applied to second impactibility scores and the third weight applied to the third impactibility scores, the first weight related to the second weight based on the first ratio.

14. The system of claim 12, wherein the processor is configured to:
calculate each of the second impactibility scores based on a gain in saved health units for a respective one of the subareas and for a number of episodes; and
calculate each of the third impactibility scores based on condition-specific actually observed or estimated clinically preventable cost for a respective one of the subareas and for the number of episodes.

15. The system of claim 14, wherein at least one of the second impactibility scores and the second impactibility scores is calculated based on at least one condition, the at least one condition determined to cause avoidable costs in a provider network.

16. The system of claim 11, wherein the processor is configured to:
receive information selecting a second outcome perspective;
calculate the first impactibility scores for the second outcome perspective;
designate an allocation of healthcare resources and cost for the first subarea based on the second outcome perspective, wherein the first impactibility scores are calculated for the respective subareas including the first subarea and wherein the second outcome perspective corresponds to a second ratio of healthcare resources and cost different from the first ratio.

17. The system of claim 16, wherein the processor is configured to:
compare the first impactibility scores generated for the first outcome perspective and the first impactibility scores generated for the second outcome perspective; and
select the first impactibility scores generated for the first outcome perspective.

18. A non-transitory machine-readable storage medium encoded with instructions for causing a processor to:
receive information selecting a first outcome perspective;
calculate first impactibility scores for the first outcome perspective,
determine a first subarea based on the first impactibility scores, and
output information through the interface indicative of a designation of an allocation of healthcare resources and cost for the first subarea based on the first outcome perspective, wherein the instructions are to cause the processor to calculate the first impactibility scores for respective subareas including the first subarea and wherein the first outcome perspective corresponds to a first ratio of healthcare resources and cost.

19. The medium of claim 18, wherein the instructions are to cause the processor to:
calculate second impactibility scores for respective ones of the subareas;
calculate third impactibility scores for respective ones of the subareas; and
calculate the first impactibility scores based on the second impactibility scores and the third impactibility scores.

20. The medium of claim 19, wherein the instructions are to cause the processor to:
apply a first weight to the second impactibility scores;
apply a second weight to the third impactibility scores; and
calculate the first impactibility scores based on the first weight applied to second impactibility scores and the third weight applied to the third impactibility scores, the first weight related to the second weight based on the first ratio.
